(19) United States
(12) Patent Application Publication
(10) Pub. No.: US 2008/0009517 A1
(43) Pub. Date: Jan. 10, 2008

(54) Use of nelfinavir as a radiation sensitizer

(75) Inventors: Anjali K. Gupta, Bala Cynwyd, PA (US); George J. Cerniglia, West Berlin, NJ (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: The Trustees of the University of Pennsylvania

(21) Appl. No.: 11/786,955

(22) Filed: Apr. 13, 2007

(30) Foreign Application Priority Data
Oct. 13, 2005 (US) PCT/US05/37262

(51) Int. Cl. A61K 31/435; A61P 35/00; G01N 33/53

(52) U.S. Cl. 514/307; 435/7.1

(57) ABSTRACT

The present invention relates to the sensitization of a cell to radiation. In particular, the present invention relates to the use of a protease inhibitor to sensitize a cancer cell to radiation.

### CROSS-REFERENCE TO RELATED APPLICATIONS

This application is entitled to priority under 35 U.S.C. § 119(e) to U.S. Provisional Patent Applications Nos. 60/618,470, filed Oct. 13, 2004, U.S. Provisional Patent Application No. 60/618,445, filed Oct. 13, 2004, U.S. Provisional Patent Application No. 60/618,486, filed Oct. 13, 2004, and U.S. Provisional Patent Application No. 60/618,448, filed Oct. 13, 2004, each of which is incorporated by reference herein in its entirety.

### BACKGROUND OF THE INVENTION

Radiation therapy is an effective tool for the treatment of many types of cancers, but the success of this type of treatment in ablating tumor growth is limited by the intrinsic resistance of cells to the procedure.

Radiation resistance in cells may arise due to activated oncogenes in a cell; however, this factor alone does not account for the increased radiation resistance, or “radioresistance,” in all tumor cells. For example, in tissue culture, the expression of ras oncogenes has been shown to increase radioresistance in NIH 3T3 cells (Fitzgerald et al., 1985, Am. J. Clin. Oncol. 8:517-522; Sklar et al., 1988, Science 239:645-647; Pirollo et al., 1993, Radiat. Res. 135:234-243; Samid et al., 1991, Radiat. Res. 126 244-250), rat embryo fibroblasts (McKenna et al., 1990, Int. J. Rad. One. Biol. Phys. 18:849-860; Ling et al., 1989, Radiat. Res. 120:267-279), rhabdomyosarcoma cells (Hermens et al., 1992, Cancer Res. 52:3073-3082), human osteosarcoma cells (Miller et al., 1993, Int. J. Cancer 53:302-307; Miller et al., 1993, Int. J. Radiat. Biol. 64:547-554) and mammary carcinoma cells (Bruyneel et al., 1993, Eur. J. Cancer 29A: 1958-1963). In contrast, the presence of K-ras in rat kidney epithelial cells rendered these cells more sensitive to radiation (Harris et al., 1990, Somat. Cell & Molec. Genet. 16:39-48). Further, human mammary epithelial cells also exhibited no increase in radioresistance in the presence of ras (Alapetite et al., 1991, Int J. Radiat. Biol. 59:385-396).

The ras oncogene may be associated with resistance of some cells to radiation (McKenna et al., U.S. Patent Application Publication No. 2002/0034725 A1, Mar. 21, 2002). The presence in cells of oncogenes other than ras, and which are involved in the ras signaling pathway, may also be associated with resistance of cells to radiation. Such oncogenes include raf (Kasid et al., 1989, Science 243:1354-1356; Pirollo et al., 1989, International Journal of Radiation Biology 55:783-796), mos (Pirollo et al., 1989, supra; Suzuki et al., 1992, Radiation Research 129:157-162), ets, and sis (Pirollo et al., 1993, supra).

Some ras mutations may result in cell transformation and other ras mutations may not result in cell transformation. Mutations in ras which result in the formation of tumors are those which give rise to an activated form of ras protein, which protein promotes transformation of the ras-expressing cell and therefore, the formation of tumors derived therefrom. Mutations in H- and K-ras are frequently found in human tumors of both epithelial and mesenchymal origin (Bos, 1989, Cancer Res. 49:4682-4689). H-ras mutations have been detected in as many as 45% of bladder cancers with the greatest occurrence in higher grade malignancies (Czerniak et al., 1992, Human Pathol. 23:1199-1204). H-ras mutations are also seen in thyroid (Lemoine et al., 1989, Oncogene 4:159-164), head and neck cancers (Anderson et al., 1992, J. Otolaryngol. 21:321-326), and sarcomas (Wilke et al., 1993, Modem Pathol. 6:129-132; Bohle et al., 1996, Am. J. Pathol. 148:731-738, 1996), prostate (Konishi et al., 1995, Am. J. Pathol. 147:1112-1122; Watanabe et al., 1994, Int. J. Cancer 58:174-178) and cervical (Riou et al., 1988, Oncogene 3:329-333) carcinomas. Mutations in K-ras have an even higher prevalence in human tumors, occurring in 75-95% of pancreatic cancers (Smit et al., 1988, Nucleic Acids Res. 16:7773-7782; Capella et al., 1991, Environ. Health Perspec. 93:125-131) and 50% of colorectal tumors (Capella et al., 1991, supra; Vogelstein et al., 1988, New Engl. J. Med. 319:525-532). A significant incidence of K-ras mutations has also been reported in adenocarcinoma of the lung (Husgafvel-Pursiainen et al., 1995, J. Occup. Environ. Med. 37:69-76), later stage cervical tumors (III and IV) (Symonds et al., 1992, Eur. J. Cancer 28A:1615-1617; Hiwasa et al., 1992, Eur. J. Gynaec. Oncol. 13:241-245) and prostate tumors (Konishi et al., 1995, supra; Watanabe et al., 1994, supra).

Mutations in membrane tyrosine kinases such as Ras and epidermal growth factor receptor (EGFR) are frequently seen in human cancers, and have been shown to result in resistance to radiation through a phosphatidylinositol-3-kinase (PI3K)-dependent pathway (Gupta et al, 2001, Cancer Res 61:4278-82; Liang et al, 2003, Mol Cancer Ther 2:353-60). There are currently clinical trials in progress that are directed to down-regulating EGFR and Ras in an effort to improve survival. Although clinical responses to EGFR inhibitors and HER-2 inhibitors, for example, correlate with high levels of receptor expression, a significant subset of patients with high receptor levels appear to be refractory to treatment.

With trials involving Trastuzumab (HERCEPTIN) as first line of treatment in metastatic breast cancer, the response rate is only 25% (Vogel et al., 2001, Oncology 61(S2):37-42). Similarly, inhibiting EGFR with anti-epidermal growth factor receptor chimeric antibody C225 has shown response rates of only about 15% in phase I clinical trials (Baselga et al, 2000, J. Clin. Oncol. 18:904-914). Iressa (gefitinib), a small molecule inhibitor of EGFR which was recently approved as third line therapy in patients with non-small cell lung carcinoma, also only has about a 10% radiographic response rate in a randomized phase II trial (Kris et al, 2003, JAMA 290:2149-58). One possible explanation for these low response rates may be that the patients also have mutations in downstream targets of growth factors and, therefore, modulating the upstream components would have little effect on outcome in patients with downstream mutations.

Cancer cells are more susceptible to damage with ionizing radiation than normal tissue. It is this differential that allows treatment of cancers with radiation, without excessively increasing the risk of side effects. For example, protocols using combinations of DNA-damaging agents, such as certain chemotherapies, with radiation have been developed. However, complications increase in conjunction with response rates. In an effort to keep the response rates high and complications low, molecular-targeted therapies have been developed, focusing on target receptor kinases that are known to be mutated in cancer cells. Although such targets initially appeared to be promising, the actual data has thus far been disappointing.

Therefore, there is an acute need to provide improvements to the radiation-based approach to treatment of cancer in order to increase the efficiency and success of the technique. Specifically, there is a need to improve the effectiveness of radiation treatment in cancer patients, and in particular, those patients refractory to currently available treatments. The present invention satisfies this need by addressing and exploiting the potential role of modulation of downstream targets in receptor pathways.

### SUMMARY OF THE INVENTION

The present invention includes a method of sensitizing a cell to radiation, the method comprising contacting a cell with a composition comprising nelfinavir. In one embodiment, the cell is a cancer cell. In another embodiment, the cell is a tumor cell.

In one embodiment of the invention, sensitization involves the inhibition of a PI3K polypeptide. In another embodiment, sensitization involves the inhibition of an Akt polypeptide. In yet another embodiment, sensitization involves the inhibition of the phosphorylation of an Akt polypeptide.

In one aspect of the invention, a cell is sensitized in vivo. In another embodiment, a cell is sensitized in vitro.

In an embodiment, the invention includes a method of identifying a compound that sensitizes a cell to radiation, wherein the compound inhibits the phosphorylation of Akt. The method comprises contacting a cell with a test compound, wherein a lower level of sensitivity to radiation of a cell contacted with the test compound compared with the level of sensitivity to radiation of a second otherwise identical cell not contacted with the test compound is an indication that the test compound sensitizes a cell to radiation.

In another embodiment, the invention includes a method of identifying a compound that sensitizes a cell to radiation, wherein the compound inhibits Akt. The method comprises contacting a cell with a test compound, wherein a lower level of sensitivity to radiation of a cell contacted with the test compound compared with the level of sensitivity to radiation of a second otherwise identical cell not contacted with the test compound is an indication that the test compound sensitizes a cell to radiation.

In yet another embodiment, the invention includes a method of identifying a compound that sensitizes a cell to radiation, wherein the compound inhibits PI3K. The method comprises contacting a cell with a test compound, wherein a lower level of sensitivity to radiation of a cell contacted with the test compound compared with the level of sensitivity to radiation of a second otherwise identical cell not contacted with the test compound is an indication that the test compound sensitizes a cell to radiation.

The invention also includes a method of reducing the growth of a tumor in a mammal, the method comprising contacting a tumor with a composition comprising nelfinavir, and irradiating the mammal. The invention further includes a method of eliminating a tumor from a mammal, the method comprising contacting a tumor with a composition comprising nelfinavir, and irradiating the mammal.

### BRIEF DESCRIPTION OF THE DRAWINGS

FIG. 1 is a table illustrating the percent occurrence of over-expression or mutation of oncogenes in various types of cancer.

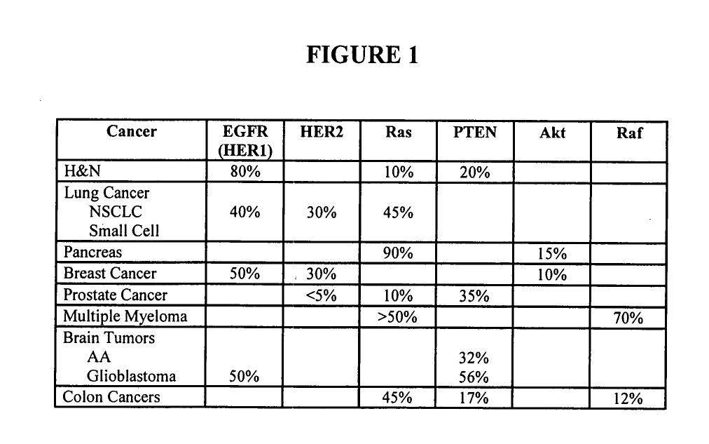

FIG. 2 is a flowchart illustrating the ras pathway and points in the pathway for which there are known inhibitors and/or therapies.

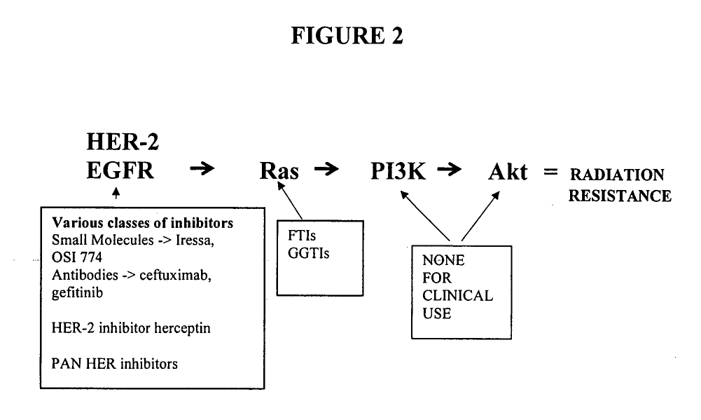

FIG. 3 is series of images of Western blots illustrating the effect of nelfinavir on Akt phosphorylation.

FIG. 4 is a graph illustrating the surviving fraction of cells in the presence and absence of nelfinavir, as a function of radiation dose.

FIG. 5 is a graph depicting the plating efficiency of SQ20B cells in the presence and absence of nelfinavir, as a function of radiation dose.

FIG. 6 is a graph depicting the plating efficiency of SQ20B cells in the presence and absence of nelfinavir, as a function of radiation dose.

FIG. 7 is an image of a Western blot demonstrating that human HIV patients taking nelfinavir demonstrate lower levels of Akt phosphorylation in white blood cells than do HIV patients not taking nelfinavir.

### DETAILED DESCRIPTION OF THE INVENTION

Radiation therapy, or irradiation of cancer cells, is the primary method for the treatment of cancer, and in particular, of tumors. Such methods involve the use of high-energy particles or radiation, including gamma- and x-rays. Because of the efficacy of radiation therapy, this therapy is used both alone and in conjunction with other forms of cancer therapy. However, one aspect of radiation therapy damages healthy cells as well as cancer cells, and therefore, methods of radiation therapy in which smaller doses of radiation can be administered to a patient are desirable. The present invention addresses this need. In particular, the present invention provides methods for the sensitization of cells to radiation, including cells that are resistant to radiation, such that smaller doses of radiation can be used to obtain equal or superior results when compared to traditional methods of irradiating cells.

Identification of a common cellular signal downstream from ras in the ras pathway: 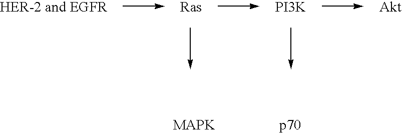
that can lead to radiation resistance is a critical step regulating tumor cell radiosensitivity in multiple human tumors (Gupta et al., 2001, Cancer Research 61:4278-4282; Gupta et al, 2003, Int. J. Rad. One. Biol. Phys. 56:846-853). Additionally, it has been shown that PI3K activation both in vitro and in vivo is related to tumor cell radiosensitivity in multiple human tumors (Gupta et al., 2001, Cancer Research 61:4278-4282; Gupta et al, 2003, Int. J. Rad. One. Biol. Phys. 56:846-853). Until now, however, there were no known clinically useful inhibitors of PI3K and/or PI3K activity.

As described elsewhere herein, one thing that has become clear from the studies involving mutated receptor kinases in cancer cells is that multiple signaling pathways often converge on a single node, the downstream component Akt. Akt is an immediate downstream target of PI3K. Therefore, in one aspect of the present invention, it will be understood that there are many points of mutation up-stream of Akt that can affect radiosensitivity, potentially enabling cells to adapt to up-stream mutations. In one embodiment, the present invention provides methods for the sensitization of cells to radiation, including cells that are resistant to radiation, by inhibiting Akt. This is because it has been shown herein for the first time that by specifically targeting Akt, the response rate to radiation can be kept high while keeping normal tissue toxicity low.

Akt is an immediate downstream target of PI3K. It has been found that that Akt activation (phosphorylation) is decreased in response to insulin after HIV protease inhibitor (HPI) treatment (Ben-Romano et al., 2003, AIDS 17:23-32). HPIs can cause insulin resistance and diabetes, and because it is known that Akt signaling plays a role in insulin signaling, the observed side-effects of HPIs may be due to interference with Akt signaling. The present invention demonstrates, for the first time, that HPI inhibitors inhibit Akt signaling in tumors.

Further, it has been shown herein for the first time that HPI-mediated inhibition of Akt signaling sensitizes a cell to radiation. That is, treatment of a cell with an HPI sensitizes the cell to radiation. Therefore, the present invention provides methods of sensitizing a cell, and in particular, a tumor cell, to radiation therapy. Such sensitization provides benefits to a cancer patient such as, but not limited to, decreasing the amount of radiation required for successful radiation therapy, decreasing the time course required for successful radiation therapy, decreasing the intensity of radiation required for successful radiation therapy, and decreasing damage to non-cancerous cells in connection with radiation therapy.

Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

Standard techniques are used for nucleic acid and peptide synthesis. The techniques and procedures are generally performed according to conventional methods in the art and various general references (e.g., Sambrook and Russell, 2001, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

The nomenclature used herein and the laboratory procedures used in analytical chemistry and organic syntheses described below are those well known and commonly employed in the art. Standard techniques or modifications thereof, are used for chemical syntheses and chemical analyses.

The articles “a” and “an” are used herein to refer to one or to more than one (i.e., to at least one) of the grammatical object of the article. By way of example, “an element” means one element or more than one element.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

As used herein, to “alleviate” a disease, disorder or condition means reducing the severity of one or more symptoms of the disease, disorder or condition.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

A “portion” of a polynucleotide means at least about twenty sequential nucleotide residues of the polynucleotide. It is understood that a portion of a polynucleotide may include every nucleotide residue of the polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

By the term “specifically binds,” as used herein, is meant a compound, e.g., a protein, a nucleic acid, an antibody, a drug molecule, a small chemical moiety, and the like, which recognizes and binds a specific molecule, but does not substantially recognize or bind other molecules in a sample.

As used herein, to “treat” means reducing the frequency with which symptoms of a disease, disorder, or adverse condition, and the like, are experienced by a patient.

As the term is used herein, “modulation” of a biological process refers to the alteration of the normal course of the biological process. For example, modulation of cancer may involve inhibition of the oncogenic process. Alternatively, modulation of cancer may involve stimulation of the oncogenic process. Similarly, “modulation” of any process or interaction is also encompassed by the present invention.

By the term “reduction of tumor growth” or “reducing tumor growth” as used herein, means a reduction in the rate of growth of a tumor or a reduction in the overall size of a tumor when the tumor has been administered the inhibitor of the invention combined with radiation, when the rate of growth of or the size of the tumor is compared with the rate of growth of or the size of a tumor which has not been administered the inhibitor.

By the term “elimination of a tumor” or “ablation of a tumor” as used herein, means that the presence of the tumor in an animal cannot be detected using ordinary tumor detection technology known in the art at the time of the present invention.

The terms “inhibition of a gene product” and “inhibition of a polypeptide” as used herein, means inhibition of the activity of a polypeptide. For example, when the polypeptide is an enzyme, inhibition of the activity of the polypeptide means inhibition of enzyme activity. The term should not be construed to mean, complete inhibition of the activity of the polypeptide. Rather, the term should be construed to mean that the level of activity of the polypeptide is reduced either partially or completely in the presence of the inhibitor of the polypeptide, compared with the level of activity of the polypeptide in the absence of the inhibitor.

By the term “in an amount sufficient to effect inhibition of a gene product” and “in an amount sufficient to effect inhibition of a polypeptide” as used herein as it refers to an inhibitor, is meant a concentration of inhibitor which inhibits the activity of a polypeptide as defined herein.

By the terms “conferring radiation sensitivity on cells” and “sensitizing a cell to radiation” as used herein with respect to the properties of a particular compound, is meant that cells are rendered more sensitive to the effects of radiation in the presence of the compound than in the absence of the compound, or in the alternative, that the cells are rendered more sensitive to the effects of radiation after treatment with the compound than prior to treatment with the compound.

As used herein, a signaling pathway is said to “involve Akt” when the signaling pathway flows to and/or through Akt, or otherwise involves interaction with or the activity of Akt. That is, the signal transmitted through or by the pathway requires Akt for the signal to be transmitted. By way of a non-limiting example, the following ras signaling pathway is said to “involve Akt”:

EGFR→Ras→PI3K→Akt;

because the pathway involves interaction with Akt.

Similarly, as used herein, a signaling pathway is said to “involve PI3K” when the signaling pathway flows to and/or through PI3K, or otherwise involves interaction with or the activity of PI3K. That is, the signal transmitted through or by the pathway requires PI3K for the signal to be transmitted. The above pathway therefore also involves PI3K.

Description

A. Methods of Sensitizing Cells to Radiation and Sensitizing Cells to Cell Death by DNA Damage

It has been discovered in the present invention that protease inhibitors can sensitize a cell to radiation. That is, treatment of a cell with a protease inhibitor prior to radiation treatment of the cell renders the cell more susceptible to DNA damage and cell death than the cell would be in the absence of any treatment with a protease inhibitor. In one aspect, the cell is treated with at least one protease inhibitor.

Additionally, it has been discovered in the present invention that compounds which inhibit the function of selected gene products render cells more susceptible to radiation. In particular, it has been discovered that compounds which inhibit modification or activation of selected polypeptides within an Akt-containing signaling pathway render cells more susceptible to radiation. In one embodiment, the signaling pathway is a ras signaling pathway. In one aspect of the invention, the polypeptide is PI3K. In another aspect of the invention, the polypeptide is Akt.

Thus, the present invention also provides a method of killing a cell, wherein a cell is administered an inhibitor of a polypeptide in combination with conventional radiation therapy. While protease inhibitors are candidate anti-tumor agents and conventional radiation is a known anti-cancer treatment, it has been discovered in the present invention that the administration of an inhibitor of a polypeptide in a ras signaling pathway to a tumor cell in combination with radiation therapy is superior in effecting death of the cell when compared with treatment of the cell with radiation alone.

The present invention also provides a method of rendering a cell more susceptible to cell death, wherein the cell is treated with a protease inhibitor prior to the treatment of the cell to cause or induce cell death. In one aspect, after the cell is treated with at least one protease inhibitor, the cell is treated with at least one compound, or at least one method, or a combination thereof, in order to cause DNA damage for the purpose of inhibiting the function of the normal cell or killing the cell.

In an embodiment, a cell is killed by treating the cell with at least one DNA damaging agent. That is, after treating a cell with a protease inhibitor to sensitize the cell to cell death, the cell is treated with at least one DNA damaging agent to kill the cell. DNA damaging agents useful in the present invention include, but are not limited to, chemotherapeutic agents (eg., cisplatinum), ionizing radiation (X-rays, ultraviolet radiation), carcinogenic agents, and mutagenic agents, among others.

In another embodiment, a cell is killed by treating the cell with at least one method to cause or induce DNA damage. Such methods include, but are not limited to, activation of a cell signaling pathway that results in DNA damage when the pathway is activated, inhibiting of a cell signaling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a protease inhibitor is administered to a cell prior to the induction of DNA damage in the cell. In another aspect of the invention, a protease inhibitor is administered to a cell concomitantly with the induction of DNA damage in the cell. In yet another aspect of the invention, a protease inhibitor is administered to a cell immediately after induction of DNA damage in the cell has begun. Methods of inducing DNA damage for the purpose of killing a cell are discussed in detail elsewhere herein.

In another embodiment, the present invention features a method of sensitizing a cell to radiation, the method comprising contacting a cell with at least one inhibitor of a non-ras polypeptide which participates in the ras signaling pathway, whereby inhibition of the polypeptide sensitizes the cell to radiation. In one aspect of the invention, a cell is a cancer cell. In another aspect of the invention, a cell is a tumor cell.

In one embodiment, the cell is in vitro. In another embodiment, the cell is in vivo.

In one embodiment of the invention, a method of sensitizing a cell to radiation includes contacting a cell with at least one inhibitor of PI3K. In one aspect of the invention, phosphorylation of PI3K is inhibited. In another embodiment of the invention, a method of sensitizing a cell to radiation includes contacting a cell with at least one inhibitor of Akt. In one aspect of the invention, phosphorylation of Akt is inhibited.

In an embodiment, the present invention features a method of sensitizing a cell to radiation, the method comprising contacting a cell with at least one protease inhibitor, wherein the protease inhibitor inhibits a non-ras polypeptide which participates in a ras signaling pathway. In one aspect, a protease inhibitor is an HIV protease inhibitor (HPI). In another aspect, the signaling pathway is a non-ras signaling pathway.

In another aspect, an inhibitor is selected from the group of inhibitors including, but not limited to, nelfinavir. Therefore, in one embodiment, the present invention features a method of sensitizing a cell to radiation, the method comprising contacting a cell with nelfinavir, whereby nelfinavir inhibits Akt, thereby sensitizing the cell to radiation. In one aspect, nelfinavir inhibits the phosphorylation of Akt.

Protease inhibitors useful in the invention include HIV protease inhibitors such as, but not limited to, AGENERASE™ (amprenavir), CRIXIVAN™ (indinavir, IDV, MK-639), FORTOVASE™ (saquinavir), INVIRASE™ (saquinavir mesylate, SQV), KALETRA™ (lopinavir and ritonavir), NORVIR™ (ritonavir, ABT-538), REYATAZ™ (atazanavir sulfate) and VIRACEPT™ (nelfinavir mesylate, NFV).

In one aspect of the invention, an HPI is used in an unmodified form. In another aspect, the HPI is modified. In the present invention, it is useful to modify an HPI for the purpose of increasing the degree of sensitization to radiation obtained when a cell is treated with the modified HPI. It will be understood that an HPI can be modified by a chemical means, a biological means, or a combination of the two. As will be understood by the skilled artisan, a modification made to an HPI can render the HPI either more effective or less effective for a particular function. Therefore, in the present invention, wherein an HPI is used to sensitize a cancer cell to radiation, it will now be understood that a modification made to an HPI will be considered beneficial in the context of the present invention if the modification has the effect of increasing the sensitivity of a cell to radiation when compared to the sensitization of a cell to radiation that is obtained when using an unmodified form of the same HPI.

Other compounds useful in the present invention include, but are not limited to, other compounds that have a same biological activity as nelfinavir. One such biological activity is the ability to inhibit the phosphorylation of Akt, as described in detail elsewhere herein. Therefore, the present invention should be understood to include a compound that inhibits the phosphorylation of Akt, and is therefore useful to sensitize a cancer cell to radiation, as set forth in detail herein. Examples of such compounds include, but are not limited to, other HIV protease inhibitors, peptidomimetics, such as those that inhibit the activity of the HIV aspartyl protease, and peptides, among others. Based on the teachings set forth herein, the skilled artisan will now know how to identify such a compound as a compound useful in the compositions and methods of the present invention.

In yet another aspect of the invention, a protease inhibitor is a non-HPI protease inhibitor. Such inhibitors include, but are not limited to, serine protease inhibitors, aspartyl protease inhibitors, cysteine protease inhibitors, metalloprotease inhibitors, aminopeptidase inhibitors, thermolysin-like protease inhibitors, and viral protease inhibitors, among others.

In one aspect of the invention, an HPI is useful to sensitize a cancer cell to radiation. In another aspect of the invention, a cell is a tumor cell. In one embodiment, the cell is in vivo. A method of the invention is thus useful for effecting reduction of tumor growth or eliminating (i.e., ablating) a tumor in an animal. Further, in order to reduce tumor growth or eliminate a tumor in an animal, the present invention demonstrates for the first time that less radiation is required to treat the animal than has heretofore been possible, thereby reducing the level of deleterious side effects experienced by the animal undergoing treatment.

In one embodiment, the invention features a method of reducing the growth of a tumor in a mammal, the method comprising contacting a tumor with at least one inhibitor of a non-ras polypeptide which participates in the ras signaling pathway, wherein the inhibitor is administered to the tumor in an amount sufficient to effect inhibition of the polypeptide, further wherein inhibition of the polypeptide sensitizes said cell to radiation. The method further comprises irradiating the mammal in order to reduce the growth of the tumor in the mammal. In one aspect of the invention, the inhibitor is nelfinavir.

In another embodiment, the invention features a method of eliminating a tumor in a mammal, the method comprising contacting a tumor with at least one inhibitor of a non-ras polypeptide which participates in the ras signaling pathway, wherein the inhibitor is administered to the tumor in an amount sufficient to effect inhibition of the polypeptide, further wherein inhibition of the polypeptide sensitizes said cell to radiation. The method further comprises irradiating the mammal in order to eliminate a tumor in the mammal. In one aspect of the invention, the inhibitor is nelfinavir.

As will be apparent from the data presented herein, the methods of the invention are applicable to multiple different types of tumors in animals including, but not limited to, solid tumors such as tumors of the prostate, lung, colon, breast, pancreas, cervical carcinoma or sarcoma, rectal tumors, ovarian tumors, bladder and thyroid tumors and head and neck tumors. Additionally, the methods of the invention are applicable to multiple different types of cancers. A cancer may belong to any of a group of cancers, examples of which groups include, but are not limited to, leukemias, lymphomas, meningiomas, mixed tumors of salivary glands, adenomas, carcinomas, adenocarcinomas, sarcomas, dysgerminomas, retinoblastomas, Wilms' tumors, neuroblastomas, melanomas, and mesotheliomas.

Animals which are administered ras protein inhibitors are irradiated in conjunction with, or subsequent to, the administration of the inhibitor. It will be appreciated that the precise protocols to be used for administration of radiation to a tumor bearing animal will depend on any number of factors including the age of the animal and the type of tumor to be treated. However, it will also be appreciated that one of skill in the art of treating tumors will know the precise protocols to be used once in possession of the present invention, the patient's tumor status and the patient's age, etc.

When irradiating an animal, generally, multiple doses of radiation are administered to the animal over a period of time in order that skin damage in the animal is minimized and the effect of the radiation on tumor growth in the animal is maximized. The rationale and methods involved in a multiple dose type radiation protocol is described in Hall (1994, Radiobiology for the Radiologist; Time, Dose and Fractionation in Radiotherapy, pp 212-229, J. B Lippincott Company, Philadelphia, Pa.) and may be used in the present invention.

In the case of specific tumor types, the protocols for irradiation may be altered to suit the specific type of tumor being treated. For example, protocols for irradiation of an animal having a colorectal tumor are described in Mohiuddin et al. (1991, Seminars, Oncology 18:411-419). Protocols for irradiation of an animal having a sarcoma are described in Delaney et al. (1991, Oncology 5:105-118). It should be noted that in the latter instance, radiation is the preferred treatment for sarcoma. Protocols for irradiation of an animal having a breast tumor are described in Mansfield et al. (1991, Seminars Oncology 18:525-535) and in Levitt (1994, Cancer 74:1840-1846). Protocols for irradiation of an animal having a head or neck tumor are described in Harari et al. (1995, Curr. Opin. in Oncol. 7:248-254). Protocols for treatment of cervical tumors are described in Perez (1993, Oncology 7:89-96) and protocols for treatment of prostate tumors are described in Perez et al. (1993, Cancer 72:3156-3173).

Preferably, the animal which is treated is a human. However, based on the disclosure set forth herein, the skilled artisan will understand that the present invention is applicable to any animal having cancer, and in particular, having a tumor.

In general, a protease inhibitor may be administered to an animal in one of the traditional modes (e.g., orally, parenterally, transdermally or transmucosally), in a sustained release formulation using a biodegradable biocompatible polymer, or by on-site delivery using micelles, gels and liposomes, or rectally (e.g., by suppository or enema) or nasally (e.g., by nasal spray). The appropriate pharmaceutically acceptable carrier, salt solution, and the like, will be evident to those skilled in the art and will depend in large part upon the route of administration.

Treatment regimes which are contemplated include a single dose or dosage which is administered hourly, daily, weekly or monthly, or yearly. Dosages may vary from 1 μg to 1000 mg/kg of body weight of the inhibitor and will be in a form suitable for delivery of the compound.

The route of administration of the inhibitor may also vary depending upon the disorder to be treated. The invention contemplates administration of an inhibitor to an animal for the purpose of treating cancer in the animal. One protocol for administration of a protease inhibitor to a human is provided as an example of how to administer a protease inhibitor to a human. This protocol should not be construed as being the only protocol which can be used, but rather, should be construed merely as an example of the same. Other protocols will become apparent to those skilled in the art when in possession of the present invention. Essentially, for administration to humans, the inhibitor is dissolved in about 1 ml of saline and doses of 1 μg, 10 μg, 100 μg or even several milligrams per kg of body weight are administered intravenously at 48 hour intervals.

An inhibitor of the present invention may be administered orally, for example, with an inert diluent or with an edible carrier. It may be enclosed in gelatin capsules or compressed into tablets. For the purpose of oral therapeutic administration, an inhibitor may be incorporated with excipients and used in the form of tablets, troches, capsules, elixirs, suspensions, syrups, wafers, chewing gums and the like. These preparations should contain a measurable amount of autoimmune inhibitor as the active ingredient, but the amount may vary depending upon the particular form and may conveniently be between about 1% to about 90% of the weight of the pharmaceutical composition. The amount of inhibitor present in compositions is such that a suitable dosage will be obtained. Preferred compositions and preparations according to the present invention are prepared so that an oral dosage unit form contains between 5.0 to 300 milligrams of an inhibitor of the invention. Dosage, in tablet or capsule form, is at a preferred dose of 1 to 25 mg/kg patient body weight per day. The dose may be increased or decreased appropriately depending on the response of the patient, and patient tolerance.

An animal having a tumor which has been administered the protease inhibitor is then irradiated following the protocols for irradiation of an animal as described in detail elsewhere herein.

B. Methods of Identifying Useful Compounds

The invention also includes a method of identifying a protease inhibitor which confers radiation sensitivity on a cell. The method comprises providing to a cell which expresses a polypeptide which participates in the ras signaling pathway a test compound. The cell is also irradiated and the level of sensitivity of the cell to irradiation is then assessed. A higher level of sensitivity of the cell to radiation in a cell administered the test compound compared with the level of radiation sensitivity in a cell which was not administered the test compound is an indication that the test compound confers radiation or to the cell. Assessment of radiation sensitivity of a cell may be accomplished using the methods described herein in the Experimental Details section, as well as those described in Carmichael et al. (1987, supra). For example, the sensitivity of the cells to radiation or chemotherapy may be assessed by measuring the extent of apoptosis of the cell population, or, alternatively, cell survival assays may be performed.

The present invention further includes a method of identifying any compound that sensitizes a cell to radiation. A method of the invention includes contacting a cell with a test compound, wherein a lower level of sensitivity to radiation of a cell contacted with a test compound compared with the level of sensitivity to radiation of a second otherwise identical cell not contacted with the test compound is an indication that the test compound sensitizes a cell to radiation. In one aspect of the invention, the test compound further inhibits PI3K. In another aspect, the test compound further inhibits Akt. In yet another aspect, the test compound further inhibits phosphorylation of Akt.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Experimental Example 1

### Phosphorylation of Akt in the Presence of Nelfinavir

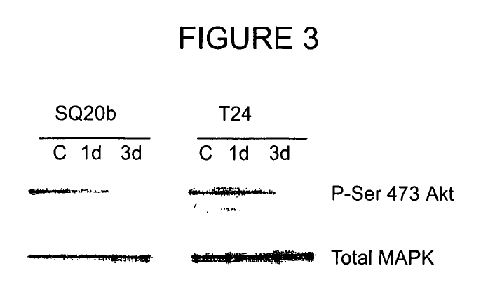

Using Western blot analysis, a human head-and-neck cancer cell line, SQ2OB, containing a constitutively-active EGFR receptor and thus, increased signaling through PI3K, was examined for Akt phosphorylation. It was found that down-regulation of Akt phosphorylation at Ser 473 was obtained in the presence of 5 μM nelfinavir. nelfinavir at a concentration of 10 μM abolished phosphorylation of Akt at Ser 473 (FIG. 3).

### Experimental Example 2

### Nelfinavir-Mediated Radiation Sensitization of Cells

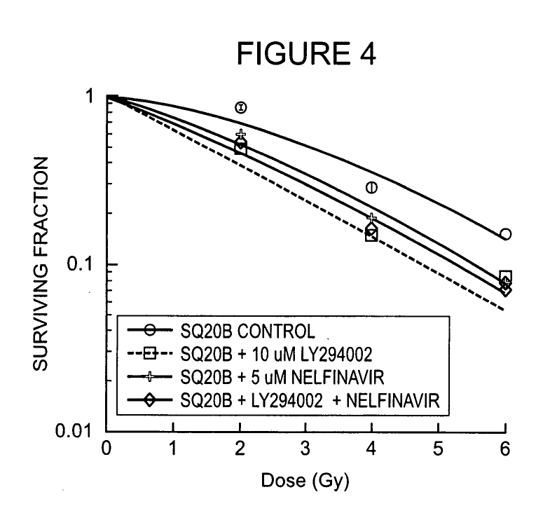

Clonogenic assays in SQ2OB cells demonstrated radiosensitization of cells after treatment of the cells with 5 μM nelfinavir. The “surviving fraction” (SF) of cells after a radiation exposure of 2 Gray (Gy) Units (ie., “SF2”) subsequently decreased from 70% to 40% after treatment with nelfinavir+2 Gy radiation (FIG. 4). These results are clinically significant, since patients treated with radiation generally receive 30+treatments of a 2 Gy dose of radiation, and the difference is thus exponentially driven.

For example, with an SF2 of 0.7 and 30 fractions of 2 Gy, the survival value would be 0.7 EE 30=2.25×10⁻⁵. If the SF2 is 0.50 and 30 fractions of 2 Gy are used, then the survival value would be 9.3×10⁻¹⁰. The survival difference translates to nearly a 5 log difference in the cell kill numbers.

### Experimental Example 3

### Nelfinavir-Mediated Radiation Sensitization of Tumors in Animals

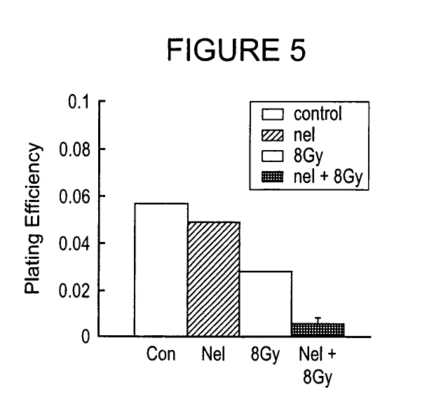

In an in vivo experiment to examine the nelfinavir-mediated radiation sensitization of tumors, tumors in animals were radiation-sensitized using nelfinavir. FIG. 5 illustrates the results of an in vivo to in vitro clonogenic assay in nude mice with SQ2OB tumors. Mice were treated with 8 Gy radiation, with and without nelfinavir at 0.6 mg/day. Nelfinavir by itself (i.e., in the absence of radiation) did not result in a change in the subsequent plating efficiency of the tumor cells derived from the mice. However, in the mice subjected to the combined treatment of radiation+nelfinavir, the treatment synergistically decreased plating efficiency of the tumor cells derived from the mice.

This result is statistically significant, as SQ2OB is generally considered to be extremely radiation-resistant tumor.

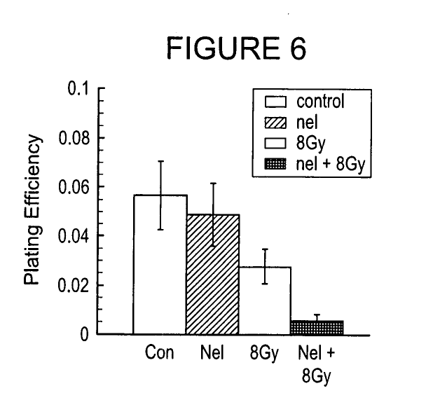

FIG. 6 illustrates the results of in vivo to in vitro clonogenic assays used to examine in vivo sensitization of tumor cells to drugs. Nelfinavir was delivered to mice at 0.6 mg/day via osmotic pump. The mice had SQ2OB xenograft tumors on hind leg. The pump was placed in mouse 2 days prior to experiment. On the day of experiment, the mouse was anesthetized and irradiated to 8 Gy. The tumor was then harvested and a cell suspension made. The cells were plated and the plating efficiency determined. The result indicted the number of cells that were viable and able to reproduce by forming colonies in each individual group (plating efficiency). Nelfinavir alone demonstrated no toxicity, but irradiation resulted in a decrease in the viability of cells. The combination of radiation and nelfinavir resulted in a significant increase in cell kill.

### Experimental Example 4

### Nelfinavir-Mediated Radiation Sensitization of Cells

Cultures of cells in log growth phase were counted and plated in 60-mm dishes containing 4 ml of media. The cells were allowed to attach and nelfinavir was added to cultures at least one hour prior to irradiation. Cells were irradiated with a Mark I cesium irradiator (J. L. Shepherd, San Fernando, Calif.) at a dose rate of 1.6 Gy/min. Colonies were stained and counted 10-14 days after irradiation. A light box was used to assist in counting colonies. The surviving fraction was calculated by dividing the number of colonies formed by the number of cells plated times plating efficiency (Table 1). Each point value represents the mean surviving fraction from at least six replicate dishes.

TABLE 1
Nelfinavir-mediated radiation sensitization of cells

| Cell Line | Control SF2 | nelfinavir SF2 |
|---|---|---|
| T24 | 0.560 +/− 0.061 | 0.452 +/− 0.023 |
| SQ20B | 0.691 +/− 0.042 | 0401 +/− 0.024 |
| MIAPACA2 | 0.906 +/− 0.086 | 0.526 +/− 0.040 |
| A549 | 0.570 +/− 0.042 | 0.410 +/− 0.062 |
| REF | 0.408 +/− 0.040 | 0.397 +/− 0.054 |

Most patients receive 30+ radiation treatments of 2Gy each. For T24 (0.56)³⁰ = 2.8 EE-8 vs. (0.37)³⁰ = 1 EE-13.

Therefore, with multiple treatments of HPI the difference translates into up to 5 logs of enhanced kill.

### Experimental Example 5

### HPI-Mediated Downregulation of Akt Phosphorylation

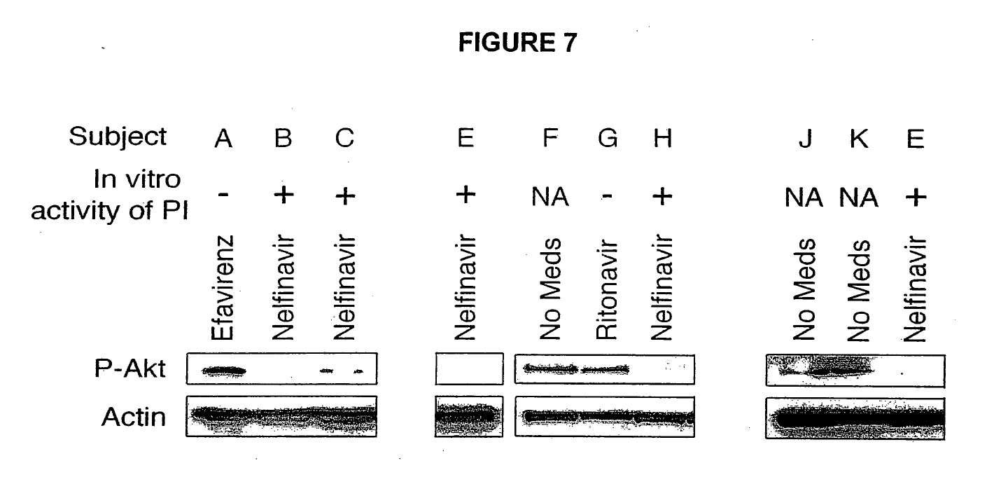

It was previously known that white blood cells (WBCs) demonstrate abundant Akt staining. As a proof of principle, it was examined whether there was down-regulation of Akt in HIV patients who are being actively treated with HPIs. Blood samples were obtained from HIV patients on courses of various combinations of drugs. WBCs were isolated and stained for Akt using a Western Blot. Patients taking nelfinavir had decreased Akt phosphorylation compared to the levels of Akt phosphorylation detected in HIV patients who were not actively using nelfinavir (FIG. 7).

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of sensitizing a cell to radiation, said method comprising contacting said cell with a composition comprising nelfinavir.

2. The method of claim 1, wherein said cell is a cancer cell.

3. The method of claim 1, wherein said cell is a tumor cell.

4. The method of claim 1, wherein said sensitization involves the inhibition of a PI3K polypeptide.

5. The method of claim 1, wherein said sensitization involves the inhibition of an Akt polypeptide.

6. The method of claim 5, further wherein said sensitization involves the inhibition of the phosphorylation of an Akt polypeptide.

7. The method of claim 1, wherein said cell is in vivo.

8. The method of claim 1, wherein said cell is in vitro.

9. A method of identifying a compound that sensitizes a cell to radiation, wherein said compound inhibits the phosphorylation of Akt, said method comprising contacting a cell with a test compound, wherein a lower level of sensitivity to radiation of said cell contacted with said test compound compared with the level of sensitivity to radiation of a second otherwise identical cell not contacted with said test compound is an indication that said test compound sensitizes a cell to radiation, thereby identifying a compound that sensitizes a cell to radiation.

10. A method of identifying a compound that sensitizes a cell to radiation, wherein said compound inhibits Akt, said method comprising contacting a cell with a test compound, wherein a lower level of sensitivity to radiation of said cell contacted with said test compound compared with the level of sensitivity to radiation of a second otherwise identical cell not contacted with said test compound is an indication that said test compound sensitizes a cell to radiation, thereby identifying a compound that sensitizes a cell to radiation.

11. A method of identifying a compound that sensitizes a cell to radiation, wherein said compound inhibits PI3K, said method comprising contacting a cell with a test compound, wherein a lower level of sensitivity to radiation of said cell contacted with said test compound compared with the level of sensitivity to radiation of a second otherwise identical cell not contacted with said test compound is an indication that said test compound sensitizes a cell to radiation, thereby identifying a compound that sensitizes a cell to radiation.

12. A method of reducing the growth of a tumor in a mammal, said method comprising:
a.) contacting said tumor with a composition comprising nelfinavir; and
b.) irradiating said mammal;
thereby reducing the growth of said tumor in said mammal.

13. A method of eliminating a tumor from a mammal, said method comprising:
a.) contacting said tumor with a composition comprising nelfinavir; and
b.) irradiating said mammal;
thereby eliminating said tumor from said mammal.
